(19) United States
(12) Patent Application Publication
(10) Pub. No.: US 2025/0057546 A1
(43) Pub. Date: Feb. 20, 2025

(54) Pedicle Fusion Device

(71) Applicant: Ariel Eli Bialik, Grand Forks, ND (US)

(72) Inventor: Ariel Eli Bialik, Grand Forks, ND (US)

(21) Appl. No.: 18/807,223

(22) Filed: Aug. 16, 2024

Related U.S. Application Data
(60) Provisional application No. 63/533,114, filed on Aug. 16, 2023.

(51) Int. Cl. A61B 17/17; A61B 17/16; A61B 17/70

(52) U.S. Cl. CPC A61B 17/1757; A61B 17/1671; A61B 17/7001

(57) ABSTRACT

Disclosed herein are novel pedicle fastener placement systems and methods for more controlled and precise fusion surgery, reducing the risks associated with spinal fixation procedures. The system includes a frame with a connected handle on one portion and connected to an insertion assembly on a second opposite end. The method includes the steps of conducting a pre-operative computed tomography scan of a patient's spine to determine an insertion point and an insertion route. The method further includes using the pedicle fastener placement system to insert a pedicle fastener at the predetermined insertion point on the pedicle, through the predetermined insertion route.

### CROSS-REFERENCE TO RELATED APPLICATIONS

The present application claims the benefit of priority to U.S. Provisional Patent Application No. 63/533,114, filed Aug. 16, 2023, which is incorporated herein by reference in its entirety.

### FIELD

The following disclosure relates to pedicle fusion systems, and more particularly, to systems and methods for inserting a pedicle fastener into a pedicle of a patient's spine.

### BACKGROUND

The field of spinal surgery involves various medical devices and systems designed to assist surgeons in the placement of fasteners, such as screws, into the pedicles of the spine. These pedicles are bony projections that come off the back posterior aspect of the vertebral body and serve as connection points for the vertebral arch. The precise placement of pedicle fasteners is critical for the stability and success of spinal fixation procedures. The risk of pedicle screw placement failure results in spinal deformities, such as degenerative hyperplasia of the posterior spinal structure and transverse process fractures. Traditional systems for pedicle fastener placement require careful alignment and positioning by the surgeon, often relying on visual and manual estimation to achieve the correct angle and depth of insertion.

### SUMMARY

The present disclosure provides to systems and methods for inserting a pedicle fastener into a pedicle of a patient's spine. A first aspect of the present disclosure presents a pedicle fastener placement system for inserting a pedicle fastener into a pedicle of a patient. The pedicle fastener placement system includes a frame configured to support a fastener driver for insertion of the pedicle fastener. The pedicle fastener placement system also includes a handle connected to a first segment/portion of the frame and configured with a plate at the end base to facilitate aligning the frame during the insertion process. The pedicle fastener placement system further includes an insertion assembly connected to a second end of the frame that is opposite the first end of the frame. The insertion assembly is configured to cause the insertion so as to insert the pedicle fastener into the pedicle of the patient.

In some embodiments, the frame includes a horizontal portion connected at a 90° angle to a vertical portion. In some embodiments, the length of the horizontal portion is adjustable to allow for calibrating the orientation of the insertion assembly. In some embodiments, the insertion assembly includes a support member pivotally connected to the second end of the frame. The support member is configured to support the fastener driving mechanism on the frame. In some embodiments, the insertion assembly further includes a fastener driver holder movably connected to a distal end of the support member.

In some embodiments, the insertion assembly further includes a fastener driver configured to be connected to the fastener driver holder. The fastener driver holder is configured to move along a portion of the support member, such that a fastener driver connected to the fastener driver holder drives a pedicle fastener into the pedicle of a patient. In some embodiments, the insertion assembly further includes an angle adjustment mechanism operably connected to the frame and operably connected to the support member. The angle adjustment mechanism is configured to control the orientation of the support member relative to the frame.

In some embodiments, the angle adjustment mechanism further includes a locking mechanism to prevent relative movement between the frame and the support member when activated, such that an angle between the frame and support member is maintained when the angle adjustment mechanism is locked by the locking mechanism. In some embodiments, the pedicle fastener is cannulated. In some embodiments, the pedicle fastener is a pedicle screw. In some embodiments, the support member of the pedicle fastener placement system includes a threaded opening oriented perpendicularly to a longitudinal axis of the pedicle fastener. In some embodiments, the fastener driver is made of a sterilizable plastic.

A second aspect of the present disclosure presents a method of inserting a pedicle fastener into a pedicle of a patient using a pedicle fastener placement system. The method includes the step of locating an entry point on the pedicle of a patient. The method also includes the step of adjusting an angle adjustment mechanism and a horizontal portion of a frame, such that the pedicle fastener placement system is oriented towards the entry point on the pedicle. The method further includes the step of moving the driver holder along the support member, forcibly causing the pedicle fastener to be inserted into the pedicle of the patient along the insertion route.

In some embodiments, the method includes the step of measuring a medial angle of a pedicle to determine an insertion route, the insertion route corresponding to a path between the entry point on the pedicle and an end point within the pedicle. In some embodiments, the method includes the step of attaching a fastener driver to the fastener driver holder of the pedicle fastener placement system. In some embodiments, the method includes the step of supporting the pedicle fastener placement system by holding a handle, such that a frame of the pedicle fastener placement system vertically aligns with a spinous process of the patient and a distal end of the fastener driver is oriented toward the entry point on the pedicle.

In some embodiments, the step of locating an entry point on the pedicle includes the step of obtaining a three-dimensional representation of the target pedicle by performing a computed tomography scan.

In some embodiments, the step of locating an entry point on the pedicle includes the step of determining a line running through the target pedicle, where the line is defined by the intersection of the coronal and sagittal planes of the target pedicle. In some embodiments, the step of locating an entry point on the pedicle includes the step of determining, as the entry point, a point defined by a posterior end of the pedicle on the line running through the target pedicle.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 illustrates a top view of a surgical environment illustrating the various components.

FIG. 2 illustrates a perspective view of a pedicle fastener placement system, in accordance with embodiments of the present disclosure;

FIG. 3 illustrates a perspective view of an angle adjustment mechanism of the pedicle fastener placement system of FIG. 2, in accordance with embodiments of the present disclosure;

FIG. 4 illustrates a perspective view of a fastener driver holder of the pedicle fastener placement system of FIG. 2, in accordance with embodiments of the present disclosure;

FIG. 5 illustrates a perspective view of a bevel gear assembly of the fastener driver holder of FIG. 3, in accordance with embodiments of the present disclosure; and

FIG. 6 is a flowchart depicting a method of operating a pedicle fastener placement system, in accordance with embodiments of the present disclosure;

While the disclosed subject matter is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

While conducting spinal surgery involving the fixation of the spine, the accurate placement of pedicle fasteners has a significant impact on the recovery of a patient. Previous approaches have included freehand techniques and the use of various guides and jigs to assist in the alignment and insertion of pedicle screws. These methods, while effective, often present challenges such as the need for extensive surgical exposure, reliance on the surgeon's experience for accurate placement, and the potential for variability in the insertion route. To address these challenges, the present disclosure provides a pedicle fusion system that offers improved accuracy and stability in the insertion of pedicle fasteners.

The disclosed system comprises a pedicle screwdriver holder device that includes an angle measurement verification system and necessary associated features. This system helps ensure that during intra-operative spinal neurosurgery fusion surgeries, the correct medical angle is maintained relative to the medial angle of the pedicle in the absence of or forgoing the more costly use of robotics equipment. Traditionally, pedicle fusion surgeries require the neurosurgeon to estimate the medial angle of the pedicle, which involves a significant level of risk and potential for surgical failure. Such failure can result in damage to the spinal cord and associated nerves if the screw does not follow the precise medial angle of the pedicle due to the inaccuracy of the neurosurgeon's estimation. By utilizing the angle measurement device for pedicle screw placement, an accurate medial angle can be achieved and maintained, thereby reducing the risks associated with manual estimation and improving surgical outcomes. Furthermore, AP and lateral image/s obtained using a C-arm, an intra-operative X-ray device, can be utilized during this process to complement the use of the medial angle verification device. The lateral image aids in confirming that the screw placement is neither cephalic nor caudal to the angle of the pedicle, thereby ensuring accurate screw pedicle placement. Such novel methods are more accurate and cost-effective than current methods utilizing solely X-ray/C-arm technology and do not entail the use of O-arm mobile CT robotics, or X-ray/C-arm robotics which are highly expensive, often unavailable in many surgical settings, and in the case of CT robotics significantly increase radiation exposure for the patient.

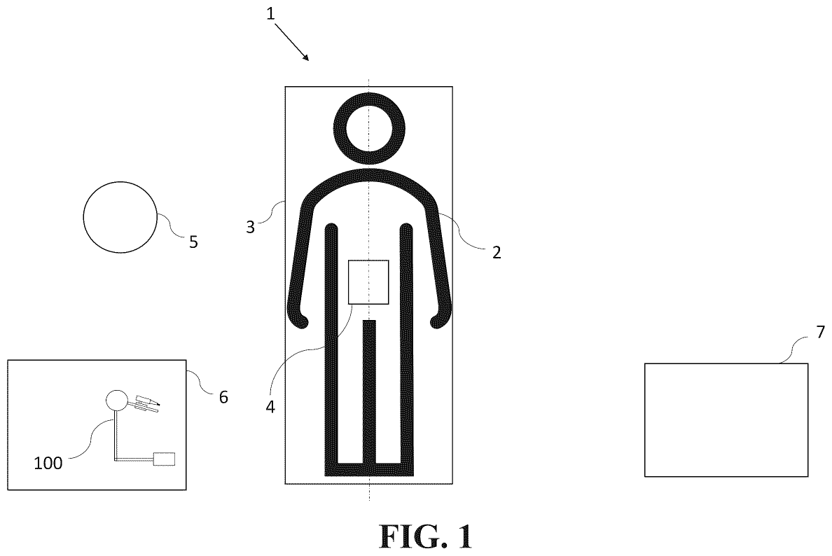

Referring to FIG. 1, surgical environment 1 comprises the patient 2 laying on a surgical table 3 in a prone position. The patients positioning is useful for ensuring access to the surgical site when operating posteriorly 4. A sterile field is maintained around the surgical table 3 to prevent contamination. The surgeon 5 is positioned near the surgical table 3, the surgical instrument table 6, and on the opposite side of the patient is often the imaging machine 7. The pedicle fusion system 100 is located on the surgical instrument table 6 among various other surgical equipment.

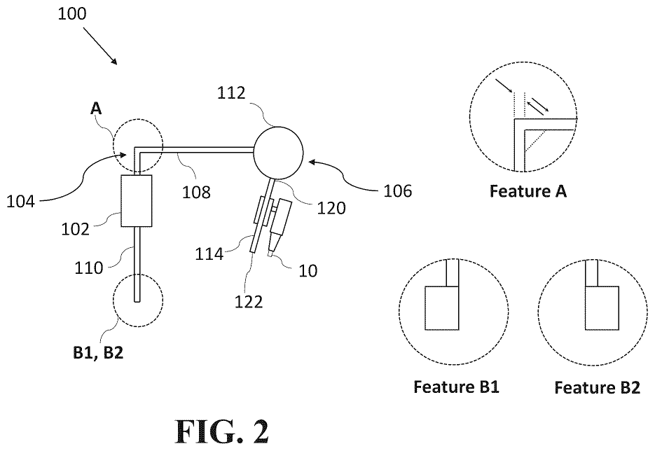

Referring to FIG. 2, pedicle fusion system 100 comprises handle 102, frame 104, and an insertion assembly 106. Frame 104 is designed to support insertion assembly 106 and facilitate the insertion of a fastener into the pedicle. Frame 104 may be constructed from high-grade stainless steel, titanium alloys, or any material suitable for surgical use known for its strength and resistance to corrosion. For lightweight alternatives, frame 104 may be constructed from advanced carbon fiber composites or polyether ether ketone materials. Frame 104 includes first portion 108 and second portion 110. The length of first portion 108 is adjustable, so as to allow for precise orientation of insertion assembly 106 towards a pedicle insertion point. In some embodiments, first portion 108 and second portion 110 are each cylindrical rods connected to one another at a right angle. Plate/spinous process aligner is connected to second portion 110 of frame and allows a surgeon to hold frame 104 in alignment to spinous process to preserve direction of medial angle during insertion of pedicle fastener 10. To ensure rigidity, handle 102 may be constructed from materials such as medical-grade stainless steel, silicone, or thermoplastic elastomers. Handle may also be tailored with ergonomic features, such as textured grips or contours that conform to the hand, reducing fatigue during prolonged surgeries.

Insertion assembly 106 is attached to first portion 108 of frame 104 and facilitates the actual insertion of the pedicle fastener 10 into the pedicle insertion point. Insertion assembly 106 includes support member 114 pivotally connected to frame 104. For uniformity and structural integrity, support member 114 may be made from the same material as frame 104 or may also incorporate lightweight materials, such as aluminum or reinforced polymers, for ease of manipulation. Insertion assembly 106 further includes angle adjustment mechanism 112, which allows for precise control of the orientation of support member 114 relative to frame 104. Angle adjustment mechanism 112 movably couples proximal end 120 of support member 114 to first portion 108 of frame 104, such that an angle between support member 114 and frame 104 can be adjusted. Angle adjustment mechanism 112 ensures that pedicle fastener 10 can be inserted at an angle that matches the pre-determined medial angle of the pedicle, as will be discussed in more detail below.

As shown at Feature A, device 100 can include an oblique angle adjustable hinge that straightens the frame 104 to 0 degrees, 90 degrees, −90 degrees, and/or 180 at the end of the second portion 110 of the frame 104. The aligner can be a plate that can be lined up alongside of posterior and caudal/downward protruding spinous process for device alignment purposes. The plate is detachable/attachable to that end of the frame for use on opposite sides. This feature can be useful for SI joint fusion purposes. When this hinge is included, the handle can be at a mid-frame position for holding. Features B1 and B2 show another perspective where plate/spinous process aligner is included at an end of the second portion 110. In Feature B1, the hinge is rotated 90 degrees from the view shown in FIG. 2 and positioned for spinous process alignment. In Feature B2, it is shown how the plate can be lined up alongside of posterior and caudal/downward protruding spinous process for device alignment purposes.

In another example, the angle adjustment mechanism 112 is modified to include a caliper. The angle adjustment caliper can turn on hinge for cephalic and caudal angle adjustments (for SI joint fusion application purposes). In this regard, the angle adjustment caliper can adjust both medially and laterally (for SI joint fusion application purposes). The caliper can include a hinge that turns cephalic and caudal has angle measurement demarcations for SI joint fusion purposes. Angles on this feature are marked at up to ten demarcations and are visible for reference and angle setting on device from each side, for example.

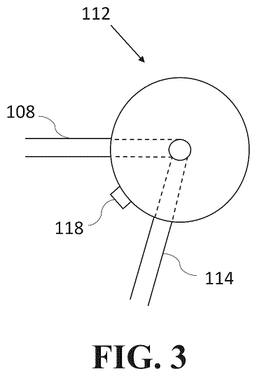

As shown in FIG. 3, angle adjustment mechanism 112 comprises locking mechanism 118, which secures the angle between support member 114 and frame 104 during the insertion process. In some embodiments, angle adjustment mechanism 112 allows for a broad range of angles by providing granular adjustments to cater to complex surgical requirements. The locking mechanism 118 can include, but is not limited to, various types such as ratchets, which allow for incremental adjustments and secure positioning, and pop pins, which provide quick and reliable locking at specific angles. Other possible locking mechanisms include thumb screws for precise manual adjustments and cam levels for fast and secure locking. By incorporating these different types of locking mechanisms, the angle adjustment mechanism 112 ensures versatility and adaptability to meet diverse surgical needs.

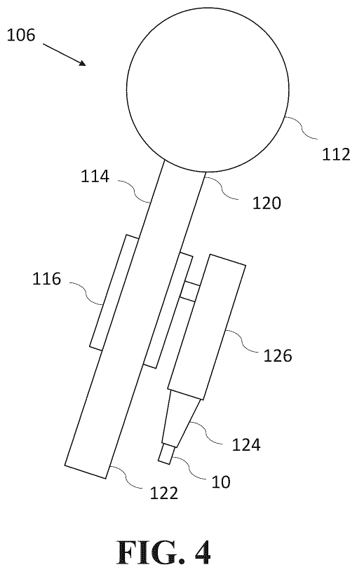

As illustrated in FIG. 4, insertion assembly 106 further comprises fastener driving mechanism 116 which is movably connected to support member 114. Fastener driving mechanism 116 facilitates the insertion of pedicle fastener 10 into a pedicle and comprises fastener driver 124 and fastener driver holder 126. Fastener driver 124 drives pedicle fastener 10, while fastener driver holder 126 serves as a connection between fastener driving mechanism 116 and support member 114. In some embodiments, fastener driver holder 126 is a cylindrical sleeve that wraps around support member 114 and is movable between proximal end 120 of support member 114 and the distal end 122 of support member 114. Fastener driver holder 126 may be constructed from a combination of metals and polymers to facilitate smooth movement along support member 114.

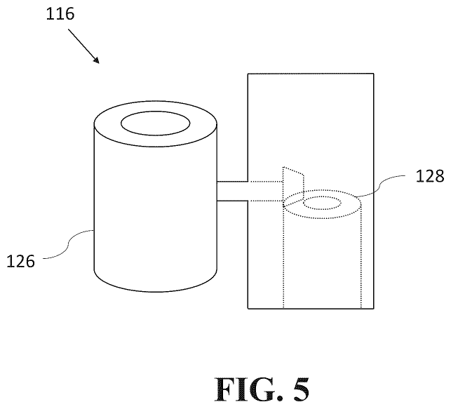

In some embodiments, fastener driving mechanism 116 includes bevel gear system 128, as shown in FIG. 5. Bevel gear system 128 allows a linear motion between fastener driver holder 126 and support member 114 to be translated into a rotational movement of fastener driver 124. Where pedicle fastener 10 is a pedicle screw and fastener driver 124 is a screwdriver, the rotational motion created by bevel gear system 128 allows the screwdriver to insert the pedicle screw into a pedicle.

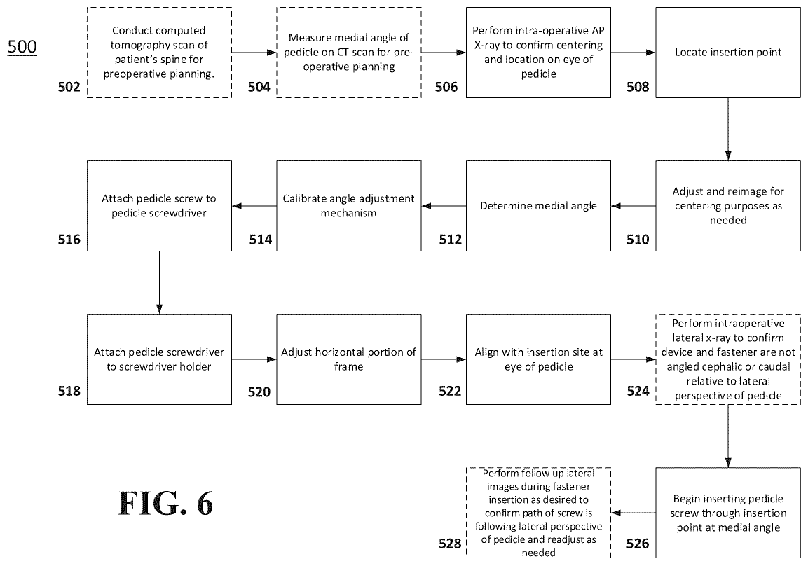

Referring to FIG. 6, method 500 provides a method of inserting a pedicle fastener into a pedicle using pedicle fusion system 100. The method begins at step 502 where a computed tomography scan is conducted on a patient before the surgical operation. Computed Tomography (CT) scans have evolved into a cornerstone of preoperative examinations in spinal surgery due to their unparalleled accuracy in three-dimensional reconstruction. The computed tomography scan provides a three-dimensional representation of the target pedicle, including the coronal, axial, and sagittal planes of the pedicle, which is fundamental to guiding spine pedicle screw placement with heightened accuracy. From preoperative computed tomography reconstructions, a localization system is derived with six parameters, including starting point height, starting point length, transverse section angle, sagittal section angle, pedicle width, and height. These parameters are then used for planning the entrance and determining the optimal orientation of pedicle screws, thereby mitigating the risk associated with their placement.

In some embodiments, the preoperative computed tomography scans may allow for the acquisition of personalized anatomical data, which is particularly beneficial for patients with abnormal anatomical structures, such as deformity or degeneration, are present. This is because the precision of computed tomography reconstruction, with accuracy down to 0.1 mm for linear data and 1° for angles, enables surgeons to conduct detailed analyses and make informed decisions regarding the most appropriate size and orientation of pedicle screws for each patient. The correlation between the starting point height, starting point length, transverse section angle, sagittal section angle, pedicle width, and height, and the anatomical variations across different segments of the spine enhance the accuracy of pedicle screw placement by maximizing the use of the pedicle's coronal and sagittal diameter, thereby ensuring the safety and effectiveness of the procedure.

At step 504, the method 500 continues by measuring a medial angle of a pedicle on the CT scan for pre-operative planning. At step 506, the method 500 includes performing an intra-operative AP X-ray to confirm centering and location on an eye of the pedicle.

With continued reference to FIG. 6, at step 508, a point on the pedicle is determined as an insertion point for pedicle fastener 10 by identifying the posterior end of a line defined by the intersection of the coronal and sagittal planes. The point at which this line meets the surface of the pedicle is selected as the insertion point. At step 510, the method 500 includes adjusting and reimaging for centering purposes as needed. At step 512, a medial angle is measured to determine an optimal insertion route for the pedicle fastener. Once the medial angle is determined, the method proceeds to step 514 where the angle adjustment mechanism 112 is calibrated so that the angle between frame 104 and support member 114 matches the medial angle. At step 516, pedicle fastener 10 (e.g., a pedicle screw) is attached to fastener driver 124. At step 518, fastener driver 124 is then attached to the fastener driver holder 126. At step 520, first portion 108 of frame 104 is adjusted to orient fastener driving mechanism 116 towards the predetermined entry point. At step 522, the method continues by aligning with insertion site at aye of pedicle.

Optionally, method 500 includes performing an intra-operative lateral x-ray to confirm device and fastener are not angled cephalic or caudal relative to lateral perspective of pedicle at step 524. Method 500 continues to step 526 where fastener driver holder 126 is moved along support member 114, resulting in the insertion of the pedicle fastener along the predetermined insertion route. Method 500 concludes at step 528 by performing follow up lateral images during fastener insertion as desired to confirm path of screw is following lateral perspective of pedicle and readjust as needed.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps can be added or omitted without departing from the scope of this disclosure. Such steps can include incorporating devices, systems, or methods or components thereof as well as what is well understood, routine, and conventional in the art. It should be noted that many alternative or additional functional relationships or physical connections can be present in a practical system. However, the benefits, advantages, solutions to problems, and any elements that can cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone can be present in an embodiment, B alone can be present in an embodiment, C alone can be present in an embodiment, or that any combination of the elements A, B or C can be present in a single embodiment; for example, A and B, A and C, B and C, or A and B and C.

In the detailed description herein, references to “one embodiment,” “an embodiment,” “an example embodiment,” etc., indicate that the embodiment described can include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Furthermore, no element, component, or method step in the present disclosure is intended to be dedicated to the public regardless of whether the element, component, or method step is explicitly recited in the claims. No claim element herein is to be construed under the provisions of 35 U.S.C. 112 (f) unless the element is expressly recited using the phrase “means for.” As used herein, the terms “comprises,” “comprising,” or any other variation thereof, are intended to cover a non-exclusive inclusion, such that a process, method, article, or apparatus that comprises a list of elements does not include only those elements but can include other elements not expressly listed or inherent to such process, method, article, or apparatus.

While various embodiments of the disclosure have been shown and described, it is understood that these embodiments are not limited thereto. The embodiments may be changed, modified and further applied by those skilled in the art. Therefore, these embodiments are not limited to the detail shown and described previously, but also include all such changes and modifications.

What is claimed is:

1. A pedicle fastener placement system for inserting a pedicle fastener into a pedicle of a patient, the pedicle fastener placement system comprising:
a frame configured to support a fastener driver for insertion of the pedicle fastener;
a handle connected midshaft the first section of the frame to a first end of the frame and configured with an aligner plate on the end to facilitate aligning the frame with the spinous process for angle verification and achievement during pedicle fastener insertion; and
an insertion assembly connected to a second end of the frame that is opposite the first end of the frame, the insertion assembly configured to cause the insertion so as to insert the pedicle fastener into the pedicle of the patient.

2. The pedicle fastener placement system of claim Error! Reference source not found., wherein the frame comprises a horizontal portion connected to a vertical portion.

3. The pedicle fastener placement system of claim Error! Reference source not found., wherein a length of the horizontal portion is adjustable to allow for orienting of the insertion assembly.

4. The pedicle fastener placement system of claim Error! Reference source not found., wherein the insertion assembly comprises a support member pivotally connected to the second end of the frame, the support member configured to support a fastener driving mechanism on the frame.

5. The pedicle fastener placement system of claim Error! Reference source not found., wherein the insertion assembly further comprises a fastener driver holder movably connected to a distal end of the support member.

6. The pedicle fastener placement system of claim 4, wherein the insertion assembly further comprises a fastener driver configured to be connected to the fastener driver holder; and
wherein the fastener driver holder is configured to move along a portion of the support member, such that the fastener driver connected to the fastener driver holder drives a pedicle fastener into the pedicle of a patient.

7. The pedicle fastener placement system of claim Error! Reference source not found., wherein the insertion assembly further comprises an angle adjustment mechanism operably connected to the frame and operably connected to the support member, wherein the angle adjustment mechanism is configured to control an orientation of the support member relative to the frame.

8. The pedicle fastener placement system of claim Error! Reference source not found., wherein the angle adjustment mechanism further comprises a locking mechanism to prevent relative movement between the frame and the support member when activated, such that an angle between the frame and the support member is maintained when the angle adjustment mechanism is locked by the locking mechanism.

9. The pedicle fastener placement system of claim Error! Reference source not found., wherein the pedicle fastener is cannulated.

10. The pedicle fastener placement system of claim Error! Reference source not found., wherein the pedicle fastener is a pedicle screw.

11. The pedicle fastener placement system of claim Error! Reference source not found., wherein the support member of the pedicle fastener placement system comprises a threaded opening oriented perpendicularly to a longitudinal axis of the pedicle fastener.

12. The pedicle fastener placement system of claim Error! Reference source not found., wherein the fastener driver comprises a sterilizable plastic.

13. A method of inserting a pedicle fastener into a pedicle of a patient using a pedicle fastener placement system, the method comprising the steps of:
locating an entry point on the pedicle;
maneuvering a driver toward the entry point on the pedicle, such that the pedicle fastener placement system is oriented towards the entry point on the pedicle; and
moving a driver holder along a support member, forcibly causing the pedicle fastener to be inserted into the pedicle of the patient along an insertion route.

14. The method of inserting a pedicle fastener of claim Error! Reference source not found., further comprising the step of measuring a medial angle of a pedicle to determine the insertion route, the insertion route corresponding to a path between the entry point on the pedicle and an end point within the pedicle.

15. The method of inserting a pedicle fastener of claim Error! Reference source not found., further comprising the step of attaching a fastener driver to a fastener driver holder of the pedicle fastener placement system.

16. The method of inserting a pedicle fastener of claim Error! Reference source not found., further comprising the step of supporting the pedicle fastener placement system by holding a handle, such that a frame of the pedicle fastener placement system and aligner plate on the end of the frame vertically aligns with a spinous process of the patient and a distal end of a fastener driver is oriented toward the entry point on the pedicle.

17. The method of inserting a pedicle fastener of claim Error! Reference source not found., wherein the step of locating the entry point on the pedicle comprises the step of obtaining a three-dimensional representation of a target pedicle by performing a computed tomography scan.

18. The method of inserting a pedicle fastener of claim Error! Reference source not found., wherein the step of locating an entry point on the pedicle comprises the steps of:
determining a line running through the target pedicle, the line defined by an intersection of the coronal and sagittal planes of the target pedicle; and
determining, as the entry point, a point defined by a posterior end of the pedicle on the line running through the target pedicle.

19. A pedicle fastener placement system for inserting a pedicle fastener into a pedicle of a patient, the pedicle fastener placement system comprising:
a frame having a horizontal portion connected at a 90-degree angle to a vertical portion, the frame configured to support a fastener driver for insertion of the pedicle fastener and to facilitate locating a pedicle insertion point for the insertion;
a handle connected to a first portion of the frame and configured to facilitate holding the frame for the insertion; and
an insertion assembly connected to a second end of the frame that is opposite the first end of the frame, the insertion assembly configured to cause the insertion of the pedicle fastener into the pedicle of the patient, the insertion assembly comprising:
a support member pivotally connected to the second end of the frame; the support member configured to support a fastener driving mechanism on the frame;
a fastener driver holder movably connected to a distal end of the support member and configured to move along a portion of the support member, such that the fastener driver connected to the fastener driver holder drives a pedicle fastener into the pedicle of a patient;
the fastener driver configured to be connected to the fastener driver holder; and
an angle adjustment mechanism operably connected to the frame and operably connected to the support member, wherein the angle adjustment mechanism is configured to control the orientation of the support member relative to the frame,
wherein a length of the horizontal portion is adjustable to allow for orienting of the insertion assembly.
